(19) United States
(12) Patent Application Publication
(10) Pub. No.: US 2023/0091552 A1
(43) Pub. Date: Mar. 23, 2023

(54) RONGEURS AND OTHER MEDICAL INSTRUMENTS

(71) Applicant: KOROS U.S.A., INC., Moorpark, CA (US)

(72) Inventors: Tibor Koros, Westlake Village, CA (US); GABRIEL KOROS, Westlake Village, CA (US)

(21) Appl. No.: 17/500,828

(22) Filed: Oct. 13, 2021

Related U.S. Application Data
(60) Provisional application No. 63/261,338, filed on Sep. 17, 2021.

(51) Int. Cl. A61B 17/16

(52) U.S. Cl. CPC A61B 17/1611

(57) ABSTRACT

Described herein are improved medical instruments, such as rongeurs. Some embodiments of the improved medical instruments include removable shafts, rotatable shafts, and mechanisms for removing and/or rotating the shafts or portions thereof. In some embodiments, the shaft has an upper and lower portion that are movable relative to one another and lock into one another using a pin-and-groove mechanism found on the inner surfaces of the upper and lower portions of the shaft.

### RELATED APPLICATIONS

This patent application claims priority to and the benefit of U.S. Provisional Patent Application No. 63/261,338 to Koros et al., filed Sep. 17, 2021, and entitled “Rongeurs and Other Medical Instruments,” the entire contents of which are hereby incorporated by reference.

The present application is also related to U.S. Des. patent application Ser. No. 29/808,294 to Koros et al., filed Sep. 17, 2021, and entitled “Rongeur,” the entire contents of which are hereby incorporated by reference.

### BACKGROUND OF THE DISCLOSURE

### Field of the Disclosure

Embodiments of the present disclosure relate to medical devices, such as rongeurs, though it is understood that the present disclosure can be applied to other devices (e.g. mechanical devices), instruments, and methods both related and unrelated to medical devices. More specific embodiments of the present disclosure relate to rongeurs with a shaft rotatable about its axis and secured by a gear-and-locking mechanism. Another specific embodiment of the present disclosure relates to medical devices, such as rongeurs, with features that improve cleanability between uses.

### Related Art

A rongeur is a hand-held surgical device used to remove a small amount of body material, such as bones or tissue. Rongeurs are typically comprised of a shaft having a lower, stationary portion with a footplate at its distal end and a cutting upper, movable portion that is longitudinally slideable relative to the stationary portion. Both the fixed portion and the movable portion are attached to a body at their proximal end. The movable portion is controlled by a trigger mechanism attached to the body.

There is typically a cutter on the distal end of the upper, movable portion that is movable towards or away from the football that is capable of grabbing or cutting anything captured between the cutter and the footplate. The trigger mechanism causes the upper, movable portion to move the cutter towards and away from the footplate of the shaft.

Instead of a cutter-and-footplate system to clamp bone or other issue, some rongeurs have a closable bite at the distal end. The fixed portion no longer has a footplate at the distal end, but is instead flat throughout, and the upper, movable portion, at its distal end, has a flat bite plate that is moveable to and away from the fixed portion, controlled by the trigger mechanism.

Because the fixed portion is typically immovable relative to the body and trigger mechanism, sometimes a surgeon needs to rotate his or her hand and/or the device in order to position the tissue or bone properly for removable. Or, a surgeon may switch out the rongeur in use for a different one that is pre-set to a different angle. This is inefficient, increasing surgery times and chances of error and infection. It also is not cost effective to have to purchase several devices with shafts set at different angles. U.S. Pat. No. 5,273,519 to Koros et al., the entire contents of which are hereby incorporated by reference, attempts to address this issue by providing for a tip that can be rotated at 45 degree increments about the axis after pressing a release lever.

Furthermore, the movement of the cutter to and from the footplate sometimes causes tissue or body fluids, such as blood, to enter the gaps between and/or throughout the movable and fix portions of the shaft and become trapped or embedded. The entrapped biomaterial is not easily removed during normal cleaning processes for medical instruments. Failure to remove this waste not only causes sanitation issues but also may compromise the device's utility. To fix this problem, some rongeurs can be completely disassembled. In particular, the shank and shaft may be completely separated from one another. However, re-assembly may be challenging for medical staff, and/or components may be lost during the cleaning process.

### SUMMARY OF THE DISCLOSURE

Described herein are improved medical devices, such as rongeurs.

One embodiment of a device according to the present disclosure comprises a rotation lock and a shaft, with the shaft including an upper portion and a lower portion. When the rotation lock is engaged, the front portion of the upper portion is held in place relative to the lower portion. When the rotation lock is disengaged, the upper portion and/or lower portion are movable relative to one another.

Another embodiment of a device according to the present disclosure includes a body, a shaft, a hinge holding the shaft to the body, and a hinge lock on the hinge and/or body. When the hinge lock is engaged, the hinge is held in place relative to the body. When the hinge lock is disengaged, the hinge is released and movable relative to the body. The hinge is movable to a position such that the shaft is removable from the body and/or hinge.

Yet another embodiment of a device according to the present disclosure includes a body, a shaft attached to the body, and a shaft lock. When the shaft lock is engaged, the shaft is nonrotatable relative to the body, whereas when the shaft lock is engaged, the shaft is rotatable relative to the body.

One embodiment of a shaft according to the present disclosure includes upper and lower portions held in place relative to one another through a pin-and-groove locking mechanism located on the surfaces of the upper and lower portions adjacent one another when the upper and lower portions are held in place relative to one another.

These and other further features and advantages would be apparent to those skilled in the art from the following detailed description.

### BRIEF DESCRIPTION OF THE DRAWINGS

FIGS. 1A-1G show various views of a first embodiment of a rongeur according to the present disclosure.

FIG. 2 shows another view of the rongeur shown in FIGS. 1A-1G.

FIGS. 3A-3G show various views of a second embodiment of a rongeur according to the present disclosure.

FIGS. 4A and 4B show views of the rongeur shown in FIGS. 3A-3G, in a second position.

FIG. 4C shows views of the rongeur shown in FIGS. 3A-3G in a third position.

FIG. 5 shows an exploded view of the rongeur shown in FIGS. 3A-3G.

FIGS. 6A-6C show various views of the shaft of the rongeur shown in FIGS. 3A-3G.

### DESCRIPTION OF THE DISCLOSURE

Embodiments of the present disclosure are directed to improved medical rongeur devices. Some embodiments of the improved medical instruments include removable shafts, rotatable shafts, and mechanisms for removing and/or rotating the shafts or portions thereof. In some embodiments, the shaft has an upper and lower portion that are movable relative to one another and lock into one another using a pin-and-groove mechanism found on the inner surfaces of the upper and lower portions of the shaft.

Embodiments of the disclosure are described herein with reference to illustrations of embodiments of the disclosure. As such, the actual size, components and features can be different, and variations from the shapes of the illustrations as a result, for example, of technological capabilities, manufacturing techniques and/or tolerances are expected. Embodiments of the disclosure should not be construed as limited to the particular shapes or components of the regions illustrated herein but are to include deviations in shapes/components that result, for example, from manufacturing or technological availability. The regions illustrated in the figures are schematic in nature and their shapes are not intended to illustrate the precise shape or functionality of a feature of a device and are not intended to limit the scope of the disclosure. In addition, components may be shown as one unit but may instead be a collection of components or units, or a collection of components or units may exist as one unit.

Throughout this description, the preferred embodiment and examples illustrated should be considered as exemplars, rather than as limitations on the present disclosure. As used herein, the term “disclosure,” “device,” “method,” “present disclosure,” “present device” or “present method” refers to any one of the embodiments of the disclosure described herein, and any equivalents. Furthermore, reference to various feature(s) of the “disclosure,” “device,” “method,” “present disclosure,” “present device” or “present method” throughout this document does not mean that all claimed embodiments or methods must include the referenced feature(s).

It is also understood that when an element or feature is referred to as being “on” or “adjacent” another element or feature, it can be directly on or adjacent to the other element or feature, or intervening elements or features may also be present. It is also understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present.

Relative terms such as “outer,” “interior,” “above,” “lower,” “below,” “horizontal,” “vertical” and similar terms, may be used herein to describe a relationship of one feature to another. It is understood that these terms are intended to encompass different orientations in addition to the orientation depicted in the figures.

Although the terms first, second, etc. may be used herein to describe various elements or components, these elements or components should not be limited by these terms. These terms are only used to distinguish one element or component from another element or component. Thus, a first element or component discussed below could be termed a second element or component without departing from the teachings of the present disclosure. As used herein, the term “and/or” includes any and all combinations of one or more of the associated list items.

The terminology used herein is for describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” when used herein, specify the presence of stated features, integers, steps, operations, elements, and/or components, but do not preclude the presence or addition of one or more other features, integers, steps, operations, elements, components, and/or groups thereof.

It is understood that the dimensions shown in the figures and described herein, as well as the ratios between those dimensions, are purely exemplary in nature. Dimensions other than those shown/described are within the scope of the disclosure, such as the shown dimensions ±5%, ±10%, ±15%, ±25%, ±50%, or other dimensions/ratios.

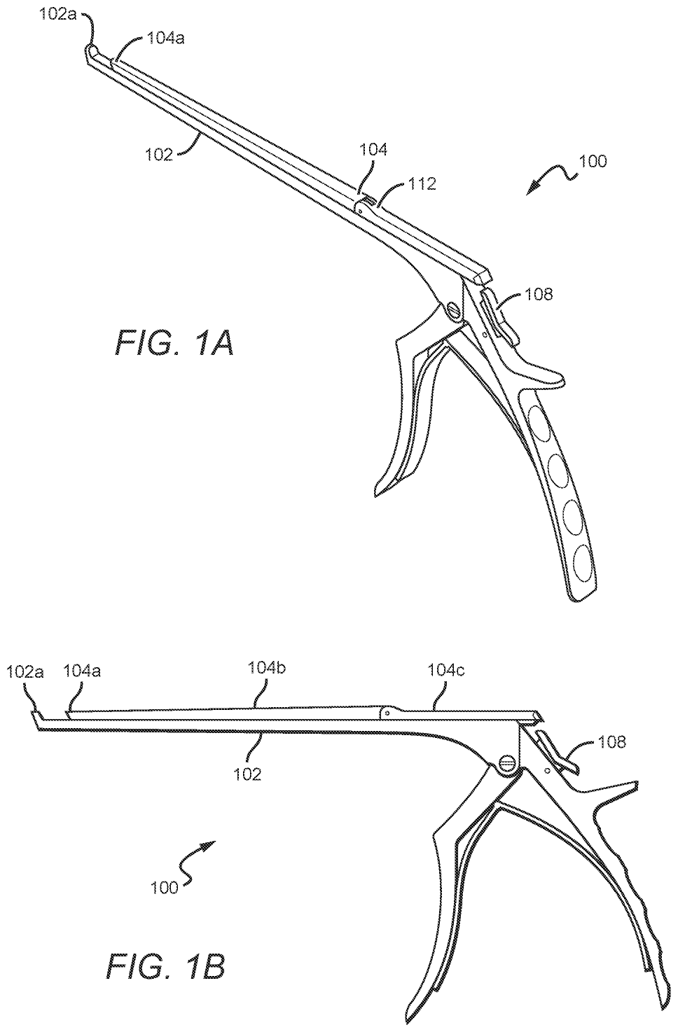

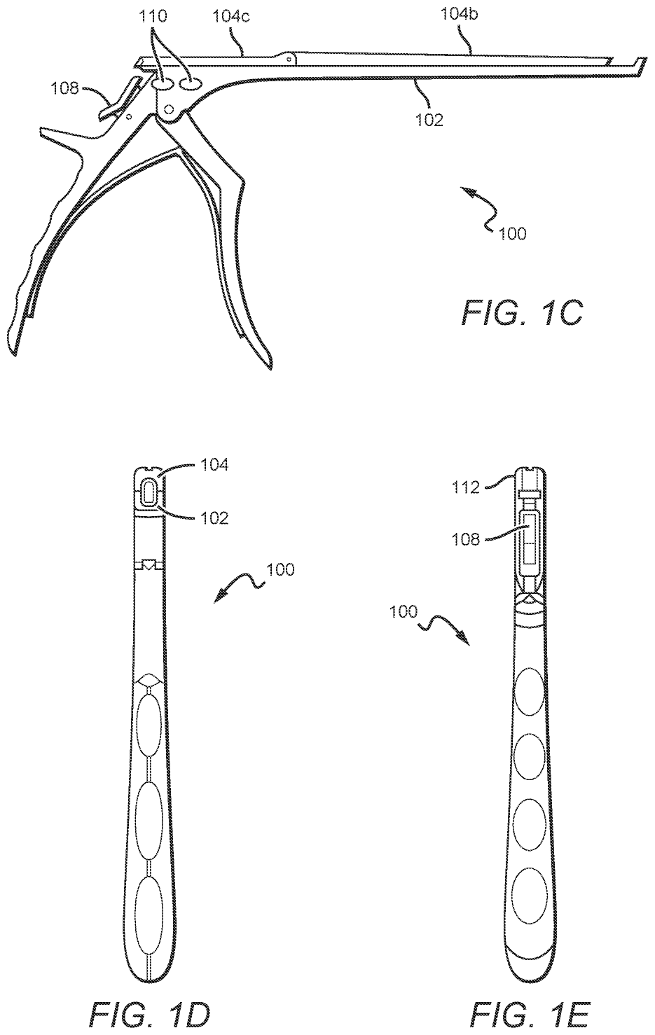

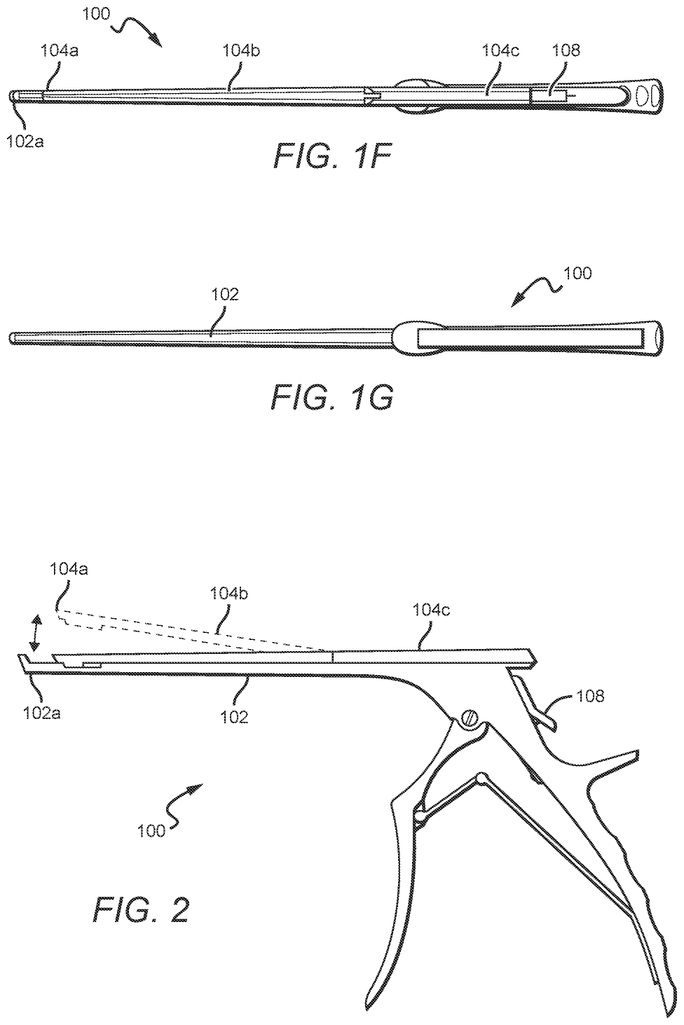

FIGS. 1A-1G and 2 show a first embodiment of a rongeur 100 according to the present disclosure. The rongeur 100 can include a shaft or rotation lock 108. The lock 108 can be biased to an engaged position, such as via elastic and/or spring biasing. When the lock 108 is disengaged (e.g., by pushing down on the lower portion thereof), a front portion 104b of the upper portion 104 of the shaft of the rongeur 100 can be rotated upwards as shown in FIG. 2, such as via a hinge/axle 112. This can be achieved, for instance, by a mechanical link between the rotation lock 108 and the front portion 104b, whereby when the lock 108 is disengaged the front portion 104b is slid backward to a position where it is no longer locked into place with the lower portion of the shaft (e.g., if the lower portion includes a block preventing rotation of the front portion 104b, the front portion 104b can be slid backwards so that the block no longer impedes rotational movement). It is understood that other locking/unlocking arrangements are possible. This can allow for easier cleaning of the newly exposed areas (e.g., the bottom of the upper portion 104 and the top of the lower portion 102). The front portion 104b of the upper portion 104 can be, for instance, 25% or more, 33% or more, 45% or more, 50% or more, 60% or more, or 75% or more of the total length of the upper portion 104. Many different embodiments and variations are possible.

Some embodiments can include one or more, two or more, and/or a plurality of viewing holes 110 on one side or both sides of the rongeur 100 as shown in FIG. 1C. The viewing holes 110 can allow the user to see into the rongeur 100 and determine if cleaning of this portion of the rongeur 100, such as internal gearing and/or other movable components, is necessary.

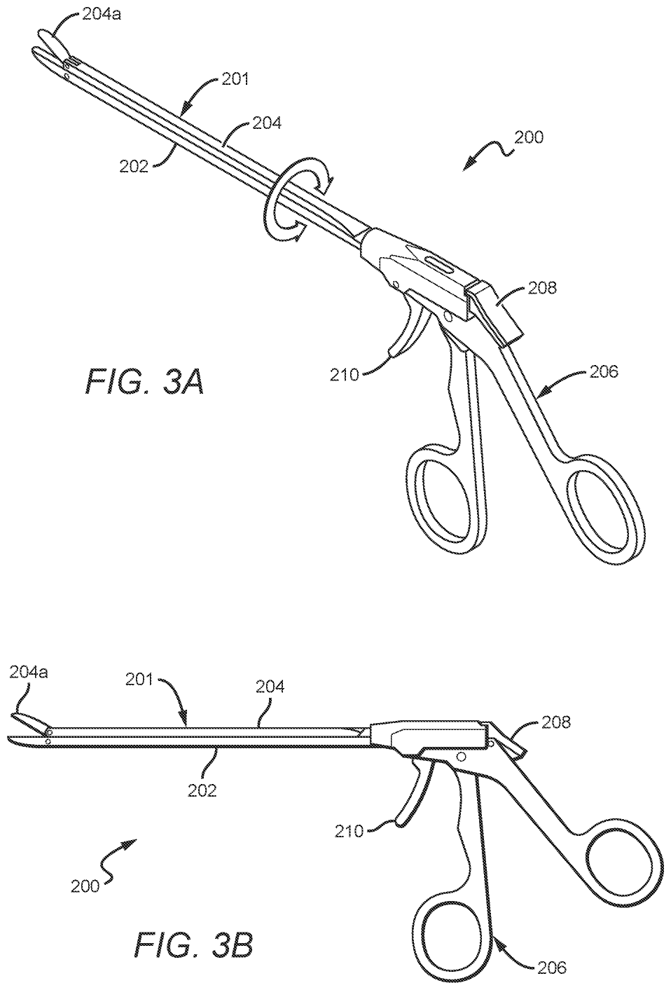

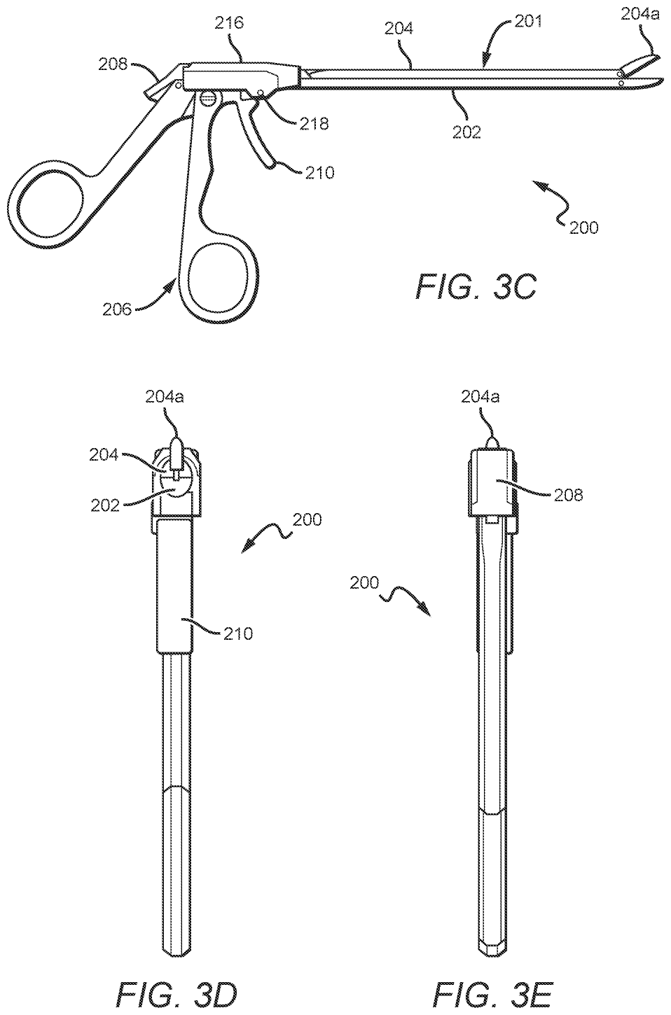

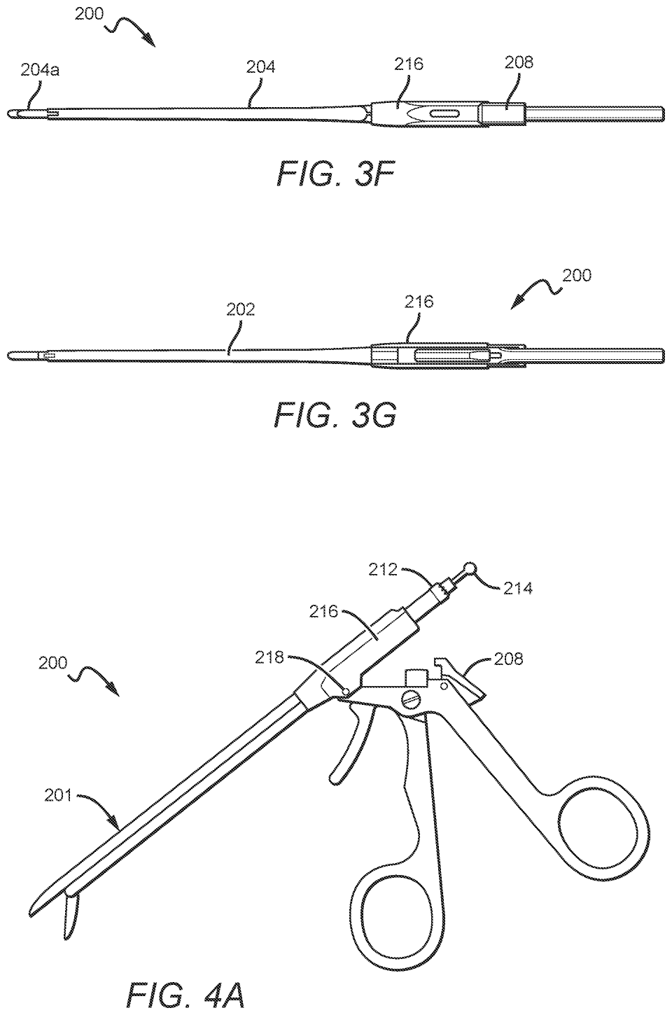

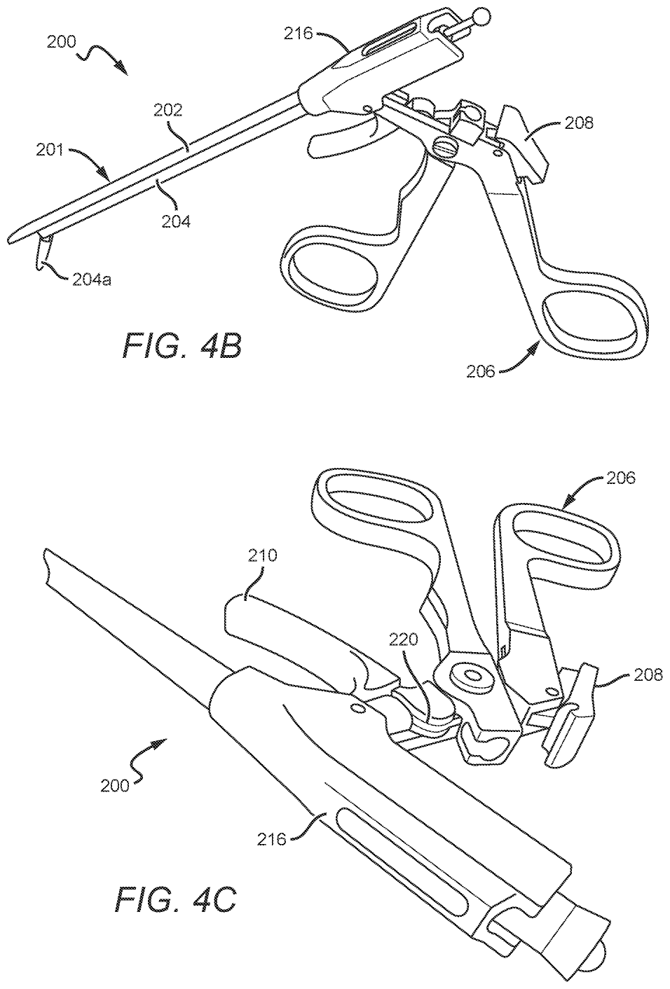

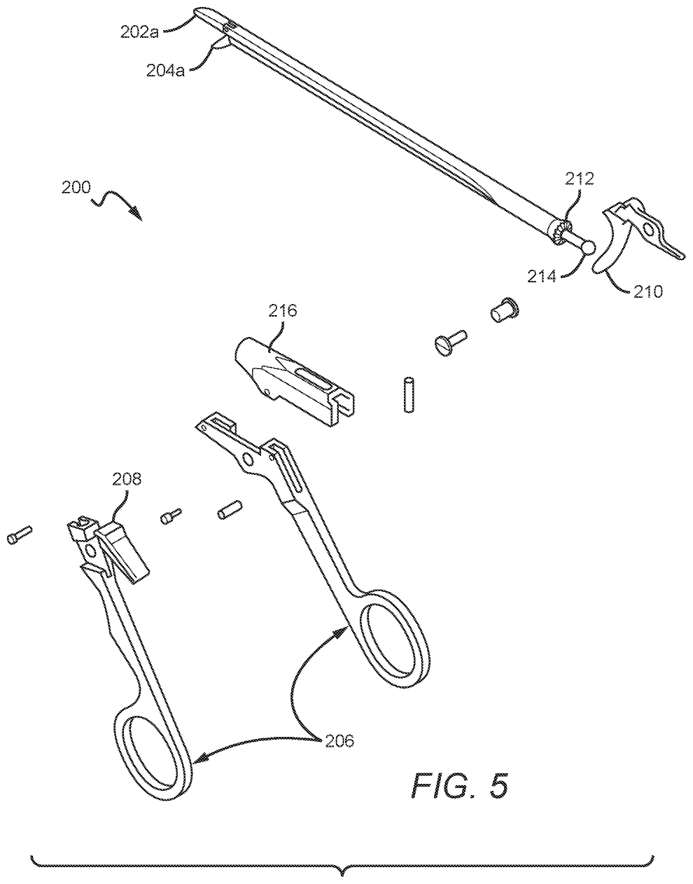

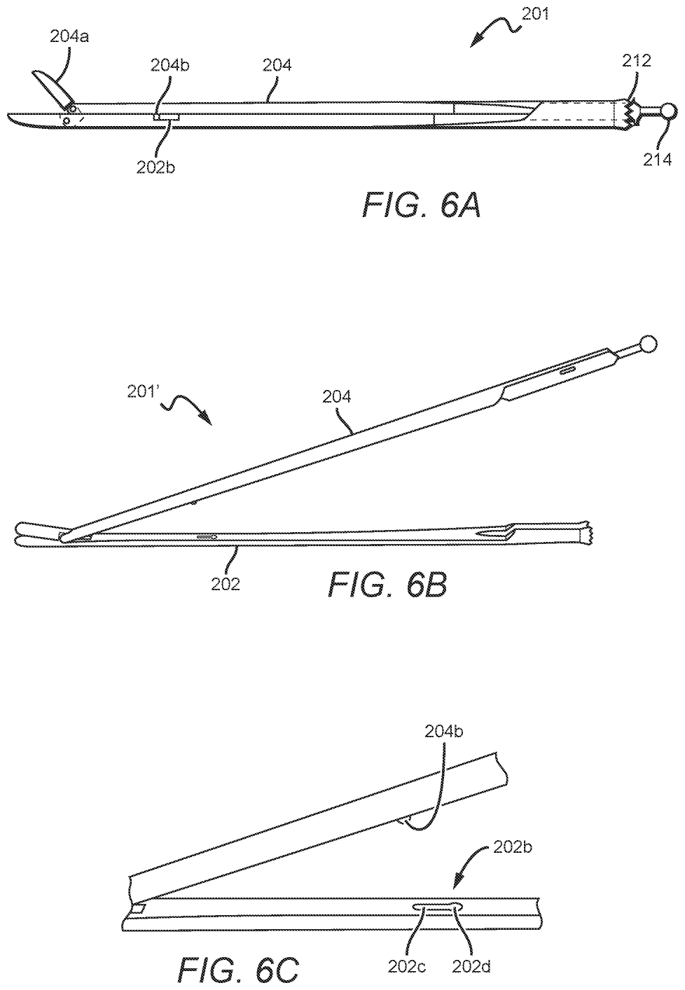

FIGS. 3A-5 show a second embodiment of a rongeur 200 according to the present disclosure. The rongeur 200 can include a hinge lock 208. The hinge lock 208 can be biased to an engaged position, such as via elastic and/or spring biasing. When the hinge lock 208 is disengaged (e.g., via pushing it down), the hinge 216 is released, the hinge lock 208 no longer impedes movement of the hinge 216, and/or the hinge 216 can be rotated (in some embodiments, along with the shaft 201 attached thereto) to a second position such as that shown in FIGS. 4A and 4B, such as about an axle 218. Once the rongeur is in such a position, the entire shaft 201 (shown in FIGS. 6A-6C) can be completely removed from the remainder of the rongeur 200, which can enhance the ability to clean the various pieces of the rongeur 200.

As best seen in FIGS. 6A-6C, once removed from the body, the first portion 204 of the shaft (e.g. an upper portion, or in another embodiment an outer portion (such as an outer tube-like structure)) can be rotated relative to another portion 202 (e.g., a lower portion, or an inner portion). In one embodiment, the rotatable portion 204 can be moved relative to the nonrotatable portion 202, such as by pulling or pushing element 214 that engages or disengages a locking mechanism prior to rotation. The locking mechanism can be disengaged by causing (e.g., by pulling on the element 214) a pin 204b located on the inner surface of one of the portions of the shaft (e.g., the upper, rotatable portion) to slide along a groove 202b located on the inner surface of the other portion of the shaft (e.g., the lower, fixed portion) and unlock the upper 204 and lower portion 202 from one another, allowing the upper portion 204 to be rotated upwards. The pin may be located on the upper portion and the groove may be located in the lower portion, or vice versa.

The groove 202b (highlighted in FIG. 6C) can comprise a first portion 202c (e.g., a channel) with a width smaller than the pin 204b, and a second portion 202d (e.g., an aperture) with a width larger than the pin 204b. Thus, when the pin 204b is within the first portion 202c the first portion 204 cannot be rotated upward, whereas when the pin 204b is within the second portion 202d it can be removed from the groove and the first portion 204 can be rotated upward

The rongeur 200 can also include a shaft lock. When the shaft lock is disengaged (e.g., via operating a trigger 210 found on the body of the device), this can allow for the shaft to be rotated, e.g. in the directions shown in FIG. 3A and/or about its own longitudinal axis. The shaft lock can then be reengaged (e.g., by releasing the trigger 210), locking the position of the shaft. Thus, the shaft can be rotated to various positions (e.g. upward facing, downward facing, sideways facing, angled facing, etc.) to enhance usability. In one embodiment, the shaft is rotatable 360°. In other embodiments, the shaft is rotatable 350° or more, 330° or more, 300° or more, 270° or more, 225° or more, 180° or more, 135° or more, 90° or more, and 180° or more but less than 360°. These are only exemplary, and many embodiments are possible.

To accomplish the rotatability of the shaft, some embodiments feature a gear 212 (or other indexing means, referred to hereinafter as a “gear” for simplicity) secured on one end of the shaft. The gear 212 can have any number of indices, such as, for instance, 4 or more indices, 6 or more indices, 8 or more indices, 10 or more indices, 12 or more indices, 18 or more indices, 6-36 indices, 8-30 indices, 12-24 indices, etc. In one specific embodiment, the gear 212 has 18 indices. In other embodiments, the gear 212 has more than 18 indices. In other embodiments, the gear 212 has less than 18 indices. These are only exemplary, and many embodiments are possible. The indices may or may not be equally spaced/equidistant from one another. The use of a gear and indices can be beneficial over embodiments with a continuous range of locking positions, because the gearing/indices combination can provide for a stronger and tighter hold on the position of the shaft compared to embodiments with a continuous range of positions where friction is primarily used to hold the shaft in place. It is understood, however, that continuous position embodiments are also possible, which can be beneficial in that they allow for a user to achieve a more exact shaft position.

Once the shaft is rotated to a position desired by the user, the shaft may be locked into place using a locking mechanism. In some embodiments, the locking mechanism, when activated, causes an indexer (e.g., a protrusion-and-aperture system) 220 to fit in between at least two of the indices on the gear 212 and hold the gear 212 in place. When the locking mechanism is activated the indices are aligned with apertures in the indexer 220 preventing the gear 212 from further rotating and locking the gear in place. In other embodiments, the locking mechanism may be created using male-female connections or relationships. In some embodiments, the locking member is active (locked) or deactivated (unlocked making the shaft rotatable) using a trigger 210 on the body 206 of the device. It is understood that many embodiments are possible.

It is understood that embodiments presented herein are meant to be exemplary. Although the present disclosure has been described in detail with reference to certain preferred configurations thereof both in the specification and in the claims, other versions are possible. Embodiments of the present disclosure can comprise any combination of compatible devices/features described herein and/or shown in the figures, and these embodiments should not be limited to those expressly illustrated and discussed. For instance and not by way of limitation, the appended claims could be modified to be multiple dependent claims so as to combine any combinable combination of elements within a claim set, or from differing claim sets. Claims depending on one independent claim could be modified so as to depend from a different independent claim. Therefore, the spirit and scope of the disclosure should not be limited to the versions described above.

While the foregoing written description of the disclosure enables one of ordinary skill to make and use what is considered presently to be the best mode thereof, those of ordinary skill will understand and appreciate the existence of variations, combinations, and equivalents of the specific embodiments, methods, systems, and examples herein. The disclosure should therefore not be limited by the above described embodiments, methods, systems, and examples. Furthermore, certain terminology has been used for the purposes of descriptive clarity, and not to limit the present disclosure. It is therefore intended that the following appended claims include all such alterations, modifications and permutations as fall within the true spirit and scope of the present disclosure. No portion of the disclosure is intended, expressly or implicitly, to be dedicated to the public domain if not set forth in the claims.

We claim:

1. A device comprising a rotation lock and a shaft, said shaft comprising an upper portion and a lower portion;
wherein when said rotation lock is engaged, a front portion of said upper portion is held in place relative to said lower portion; and
wherein when said rotation lock is disengaged, said upper portion and/or said lower portion are movable relative to one another.

2. The device of claim 1, wherein when said rotation lock is disengaged, said upper portion is rotatable relative to said lower portion.

3. The device of claim 1, wherein said rotation lock is mechanically linked to said front portion of said upper portion such that when said rotation lock is disengaged, said front portion of said upper portion is slid backward relative to said lower portion.

4. The device of claim 1, wherein said device is a rongeur.

5. A device comprising:
a body;
a shaft;
a hinge holding said shaft to said body; and
a hinge lock on said hinge and/or said body;
wherein when said hinge lock is engaged, said hinge is held in place relative to said body; and
wherein when said hinge lock is disengaged, said hinge is released and movable relative to said body, said hinge movable to a position such that said shaft is removable from said body and/or said hinge.

6. The device of claim 5, wherein a side of said shaft that connects to said hinge and said hinge lock further comprises a ball.

7. The device of claim 6, wherein said ball has a diameter smaller than the diameter of said hinge.

8. The device of claim 5, wherein said shaft comprises an upper portion and a lower portion.

9. The device of claim 8, wherein said upper and lower portions of said shaft are held in place relative to one another through a pin-and-groove locking mechanism located on the surfaces of the upper and lower portions adjacent one another when the upper and lower portions are held in place relative to one another.

10. A device, comprising:
a body;
a shaft attached to said body; and
a shaft lock;
wherein when said shaft lock is engaged, said shaft is nonrotatable relative to said body; and
wherein when said shaft lock is disengaged, said shaft is rotatable relative to said body.

11. The device of claim 10, wherein said shaft lock comprises a trigger connected to said body for disengaging said shaft lock.

12. The device of claim 10, wherein when said shaft lock is disengaged, said shaft is rotatable about its own axis.

13. The device of claim 10, wherein the portion of said shaft that connects to said shaft lock further comprises a gear with indices around an axis of the shaft.

14. The device of claim 13, wherein said shaft lock further comprises grooves that match the space between the indices on said gear, said grooves configured to lock with said gear when said shaft lock is engaged.

15. The device of claim 13, wherein said gear comprises 12 or more indices.

16. The device of claim 10, wherein said shaft is a longitudinal rongeur shaft comprising a first portion and a second portion, wherein said first portion is rotatable upwards about an axel to expose one or more surfaces of said second portion.

17. The device of claim 16, wherein said first portion is an upper portion and said second portion is a lower portion.

18. The device of claim 17, wherein said upper and lower portions of said shaft are held in place relative to one another through a pin-and-groove locking mechanism located on the surfaces of the upper and lower portions adjacent one another when the upper and lower portions are held in place relative to one another.

19. The device of claim 18, wherein said groove comprises a channel with a width smaller than said pin, and an aperture with a width larger than said pin.

20. The device of claim 19, wherein said pin-and-groove locking mechanism is unlocked by pulling said pin through said channel such that it can be removed through said aperture.
